(19) United States
(12) Patent Application Publication
(10) Pub. No.: US 2012/0123463 A1
(43) Pub. Date: May 17, 2012

(54) MECHANICALLY-GUIDED TRANSORAL BOUGIE

(76) Inventor: MOISES JACOBS, MIAMI, FL (US)

(21) Appl. No.: 12/944,286

(22) Filed: Nov. 11, 2010

(51) Int. Cl. A61M 29/00

(52) U.S. Cl. 606/191

(57) ABSTRACT

The present invention is referred to a mechanically-guided transoral bougie, comprising an elongated body with an external end and a distal end; said external end includes a guiding mechanism mechanically connected to said distal end to allow the surgeon to move said distal end in any direction once the bougie is inserted into the stomach. Said guiding mechanism includes a manually operated guiding control.

### BACKGROUND OF THE INVENTION

1. Field of the Invention

The present invention relates, in general, to surgical devices and more particularly is referred to a mechanically-guided transoral bougie comprising an elongated body with a distal end and an outer end, said outer end includes a mechanical guidance device that allows the surgeon to torque the distal end of said bougie in any direction, right, left, up or down into the stomach. Even more particularly the present invention is referred to a bougie as a dilator or calibrating tube capable of being inserted into the patient's stomach through the esophagus, which is especially useful for performing endoscopic obesity procedures as is explained in detail below.

2. Description of the Prior Art

A bougie is a thin cylinder of rubber, plastic, metal or another material that a physician inserts into or though a body passageway, such as the esophagus, to diagnose or treat a condition. A bougie may be used to widen a passageway, guide another instrument into a passageway, or dislodge an object.

Bougies are available in a wide range of sizes and degrees of flexibility. They may consist only of a simple cylinder. The cylinder may be equipped with such devices as: (1) an inflatable balloon to apply pressure against obstructions or narrowed walls; (2) a gauge to measure the pressure applied by the balloon; (3) a wire that is positioned at the site of a stricture, blockage or another problem to guide other instruments into the passageway; (4) a channel through which dye can be injected so that a site can be identified on a fluoroscope and (5) a light to illuminate a passageway for examination.

Bougies may be used to treat strictures and blockages in the esophagus, the intestines, the rectum, the anus, the urethra. To remove an object lodged in the esophagus, a lubricated bougie may be used to dislodge the object and move it down into the stomach. The object then passes through the intestines.

Bougies equipped with balloons may be used to eliminate both strictures and blockages in the esophagus. In a typical procedure, a lubricated bougie may be inserted under local anesthesia. When the bougie reaches the site of the stricture or blockage, the balloon is inflated. The pressure from the balloon then can widen a narrowed passageway.

Nowadays, bougies may also be used to perform modern surgical procedures like obesity procedures, and more particularly endoscopic bariatric surgery procedures. One example is the transoral gastroplasty (TOGA), a procedure in which a set of flexible stapling devices are delivered through a transoral bougie into the stomach, and the staples create a restrictive pouch for food. In all these procedures, the surgeon needs to get access to the stomach and insert, through the passageway defined by the bougie, different instruments or devices.

There are different types of bougies, namely:
Mercury weighted bougies: are blindly inserted bougies placed into the esophagus by the treating physician. They are passed in sequentially increasing sizes to dilate the obstructed area. They must be used with precaution in patients with narrow strictures, as they may curl proximal to the obstruction. The most commonly used mercury weighted bougies are the Maloney bougie dilators.
Bougie over guidewire dilators are used at the time of gastroscopy or fluoroscopy. An endoscopy is usually performed first to evaluate the anatomy, and a guidewire is passed into the stomach past the obstruction. This may also be done fluoroscopically. Bougies are again introduced—this time over the guidewire—in sequentially increasing sizes. The most commonly used bougie over guidewire dilators are the Savary or Savary-Guillard dilators.
Pneumatic dilatation or balloon dilatation is also typically done at the time of endoscopy or fluoroscopy. A balloon is inserted in the deflated form into the area of narrowing. It is then inflated with air to a certain pressure that is pre-set for a given circumference

All the above mentioned bougies are just different type of passageways capable of being introduced in the human body but with no capability of guiding its distal end once introduced into the stomach or any other organ.

There are several types of known bougies in the prior art. For example, U.S. Pat. No. 5,766,202 describes a wire-guided esophageal bougie comprising a a PTFE-lined, silicone rubber tube with a tapered, silicone rubber dilator on its distal end, the dilator being more flexible than the PTFE-lined tube. Radiopaque material throughout the length of the tube renders it fluoroscopically visible in the esophagus while a radiopaque ring in the dilator fluoroscopically locates the position of the dilator relative to a stricture.

Also U.S. Pat. No. 5,718,666 describes a light-conducting bougie consisting of an elongate flexible member operable for conducting light from the proximal end of the bougie to the distal portion thereof. The light-conducting bougie is made from a substantially homogenous, optically transparent polymer being flexible and operable for the transmission of light. The bougie is particularly useful for transilluminating tissue within a body.

U.S. Pat. No. 5,624,432 teaches about an illuminated bougie in which the elongate bougie body is formed of flexible light-transmitting material. A socket for detachably attaching the light-transmitting end of a fiber optic bundle is formed in the proximal end of the bougie.

U.S. Pat. No. 5,366,471 shows an esophageal dilator comprising a hollow, flexible tube filled with a dispersion of Tungsten particles in a fluid silicone material for treating cardiospasm, esophagitis, stenosis and other esophageal diseases.

Finally, U.S. Pat. No. 4,942,869 describes a mechanically-expandable urethral bougie especially useful for dilation of urethral strictures comprises a probe formed from two elongated rods connected at their one end, the rods having a semicircular cross-section and grooves at the mating surfaces. These grooves form a guide slot for a core element which has wedging elements engageable with camming surface of the rods.

None of the above described devices involves a bougie including a guiding device for moving the distal end thereof once introduced into the stomach. The main feature of said guiding device is giving to the surgeon the possibility of guiding the distal end through the stomach to the duodenum. But this is not the only feature the present invention provides. A brief summary of the improvements is:
a) The purposed bougie may have side channels and or a central channel, through which suction may be applied;
b) It may also be attached to balloons that may run alongside the bougie for inflation or deflation, for adjusting the caliber of the bougie;
c) It may be lit or coated with fluorescent material to be able to be identified intra luminally;
d) It may also be magnetized so that external magnets may be able to move it into place;
e) It might also be coated with radiopaque materials for identification by radiographs;
f) The side channels may also act as conduits for passage of instruments, fluids, or other materials;
g) It may also have a central lumen through which an endoscope may be passed;
h) The walls may be clear so as to be able to see through it; and
i) With the side balloons may also act as a stent for the esophagus and stomach in case of a gastric or esophageal leak.

As such, a mechanically-guided multi-purpose transoral bougie device for performing endoscopic surgical procedures with the possibility of guiding the distal end thereof is still desired in the market.

### SUMMARY OF THE INVENTION

A main object of the present invention is to provide a mechanically-guided transoral bougie device, capable of entering into a stomach and move the distal end thereof in any direction: left, right, down or up.

It is another object of the present invention to provide a bougie including suction channels running alongside its elongated body for inflation or deflation purposes.

Yet another object of the present invention is to provide a bougie coated with fluorescent material to be able to be identified intra-luminally.

Another object of the present invention is to provide a bougie which external wall is magnetized so that external magnets may be able to move it into place during a surgical procedure.

Yet another object of the present invention is to provide a bougie coated with radiopaque materials for identification by radiographs.

Also another aspect of the purposed invention comprises a device including a central lumen through which an endoscope may be passed.

In summary, the present invention is referred to a mechanically-guided transoral bougie, comprising an elongated body with an external end and a distal end; said external end includes a guiding mechanism mechanically connected to said distal end to allow the surgeon to move said distal end in any direction once the bougie is inserted into the stomach. Said guiding mechanism includes a manually operated guiding control.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 schematically shows a human body, the upper gastric system, and the purposed bougie inserted therein.

FIG. 2 is another schematic perspective view of a partially cut-off stomach illustrating how the distal end of the bougie faces the anterior wall of the stomach when is introduced through the esophagus, and in order to reach the duodenum the distal end thereof should be guided or maneuvered.

FIG. 3 is a general perspective view of the bougie in accordance with one of the preferred embodiment of the present invention; finally:

FIG. 4 is another schematic view of the bougie in accordance with the present invention, showing the possible moves of the distal end thereof.

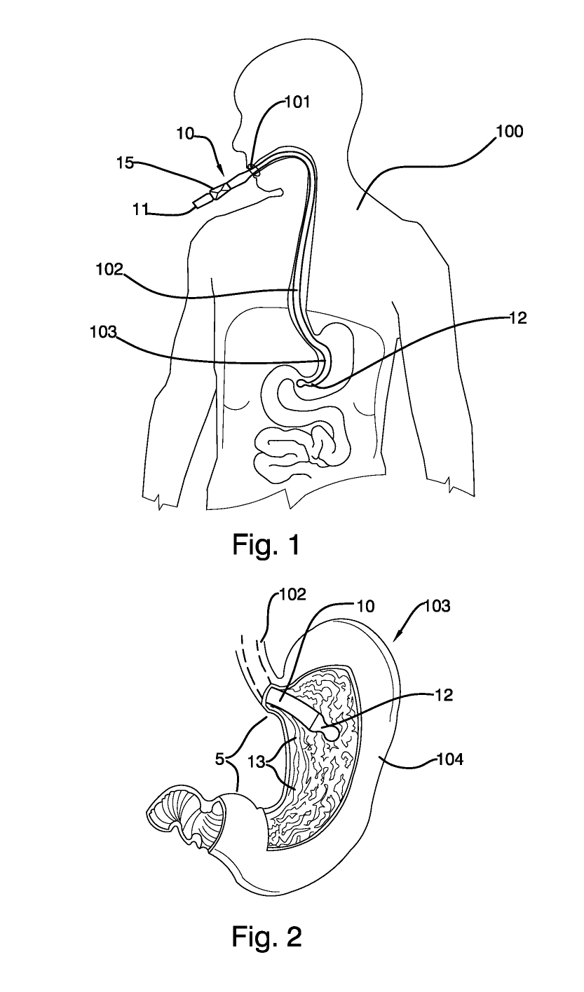

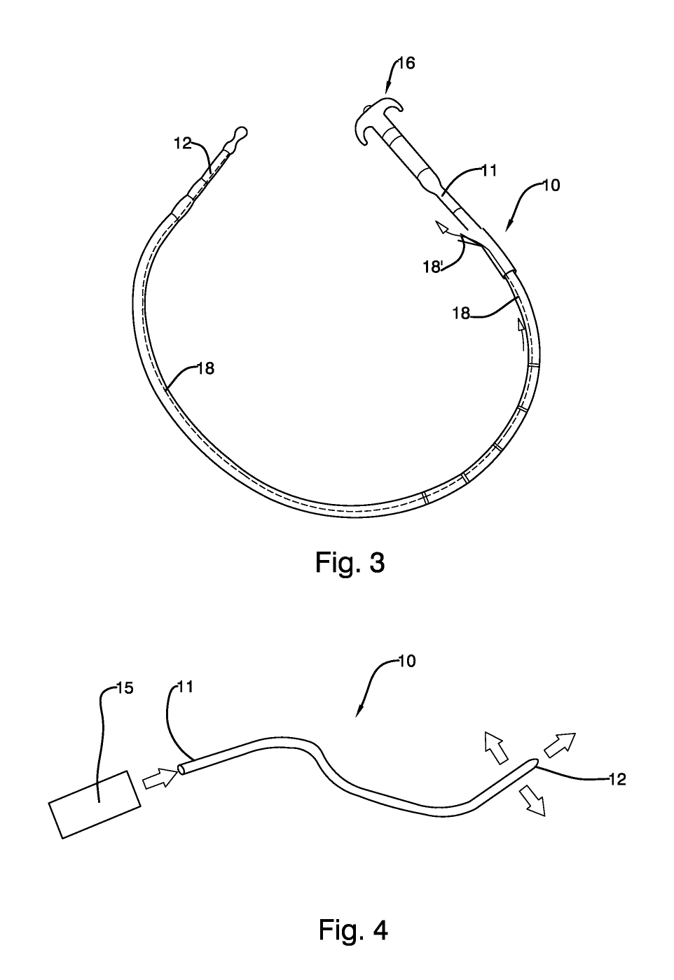

### DETAILED DESCRIPTION OF REPRESENTATIVE EMBODIMENTS

Referring now to the attached figures, the mechanically-guided transoral bougie 10, in accordance with a first embodiment, comprises an external end 11 and a distal end 12. Said bougie is normally introduced into the human body 100 through the patient's mouth 101 and the esophagus 102 until the stomach 103. As the stomach has a generally curved shape, when the distal end 12 reaches the interior of the stomach points toward the anterior face 104 thereof. Depending on the type of surgical procedure the surgeon needs to perform, said distal end should be moved upwards, downwards, leftwards or rightwards regarding the position the distal end has when reaches the interior of the stomach 103 (see FIG. 2).

At present, bougies do not have the possibility of maneuvering the distal end thereof therefore it is necessary to perform said maneuver manually. This takes time and creates cumbersome procedures that distract the surgeon and his team.

Precisely to overcome this inconvenience, the present invention provides a bougie 10 which external end 11 includes a guiding device 15. This guiding device 15 provides a controlling means 16 connected to the distal end 12 to allow the surgeon to perform a rotating or manipulative technique for moving said distal end into the stomach. In this fashion, the bougie may be directionally guided to the duodenum, or to the fundus, or any other region the surgeon may need to access.

The elongated body 13 of bougie 10 may include at least one lateral channel 18 through which suction may be applied to suck blood or the like during the procedure. Said suction channel 18 ends at the upper end of the bougie in a disposal outlet 18′. Depending on the size of the bougie, two lateral channels 18 may be included, or a central suction channel. Said side channels may also act as conduits for passage of instruments, fluids, or other materials

Even though it is not illustrated in the attached figures, the purposed bougie may also be attached to balloons that may run alongside the bougie for inflation or deflation to be able to adjust the caliber of the bougie.

In addition the bougie may be lit or coated with fluorescent material to be able to be identified intra-luminally or with radiopaque materials for identification by radiographs.

The outer walls of the bougie may also be magnetized so that external magnets may be able to move it into place. This may be obtained by manufacturing said outer walls with a magnetized material or providing some external magnetization process or device.

In some surgical procedures, an endoscope is necessary, therefore the bougie may have a central lumen through which an endoscope or gastroscope may be passed. Also its external walls may be clear so as to provide to the surgeon the possibility of being able to see through it.

Even though it is not the main purpose of the present invention, bougie 10 with the side balloons may also act as a stent for the esophagus and stomach in case of a gastric or esophageal leak. By insufflating the balloons, enough bulk and pressure may be created to keep the stent in place. Care must be taken to make sure this is a low pressure system to prevent ischemia of the walls of the organ.

It is also important to point out the possibility of decoupling the distal end of the bougie with the proximal end, such that the distal end may be left in place during the surgical procedure. There are different ways to do that. For example, the distal end could be held in place by increasing the diameter of the distal end of the bougie by insufflating low pressure balloons located either on the side of the bougie or these balloons may also be placed circumferentially. In this case, the bougie itself could act as a stent. If the bougie/stent needed to be removed, the balloons could be burst endoscopically, and the bougie extracted. Also said distal end could be made of a bioabsorbable material therefore its removal will not be required.

In the embodiment described above the bougie may act as a stent. In another embodiment, this bougie can also act as a delivery mechanism for a stent, or anastomotic device that can be draped around the outside of the bougie, or passed through the inner opening for deployment.

In the above cases when the bougie acts as a stent, it would be to divert the intestinal content, swallowed foods, and liquids to prevent leaks, or heal leaks. In addition, this may also be used to cause a bypass of nutrients for weight loss.

While the preferred embodiments of the invention have been described above, it will be recognized and understood that various modifications can be made in the invention and the appended claims are intended to cover all such modifications which may fall within the spirit and scope of the invention

I claim:

1. Mechanically-guided transoral bougie, comprising an elongated body with an external end and a distal end; said external end includes a guiding mechanism connected to said distal end to allow the surgeon to move said distal end in any direction once the bougie is inserted into the stomach.

2. Mechanically-guided transoral bougie, in accordance to claim 1, wherein the bougie includes side channels and/or a central channel, through which suction may be applied.

3. Mechanically-guided transoral bougie, in accordance to claim 2, wherein said side channels act as conduits for passage of instruments, fluids, or other materials.

4. Mechanically-guided transoral bougie, in accordance to claim 1, wherein the body of said bougie is attached to balloons running alongside for inflation/deflation, adjusting the caliber of the bougie.

5. Mechanically-guided transoral bougie, in accordance to claim 1, wherein the external surface of said bougie is coated with fluorescent material to be able to be identified intra luminally.

6. Mechanically-guided transoral bougie, in accordance to claim 1, wherein the body of said bougie is magnetized so that external magnets may be able to move it into place.

7. Mechanically-guided transoral bougie, in accordance to claim 1, wherein the body of said bougie is coated with radiopaque materials for identification by radiographs.

8. Mechanically-guided transoral bougie, in accordance to claim 1, wherein the body of said bougie has a central lumen through which an endoscope is passed.

9. Mechanically-guided transoral bougie, in accordance to claim 1, wherein the body of said bougie has clear walls so as to be able to see through it.

10. Mechanically-guided transoral bougie, in accordance to claim 1, wherein the body of said bougie acts as a stent for the esophagus and stomach in case of a gastric or esophageal leak.

11. Mechanically-guided transoral bougie, in accordance to claim 1, wherein the distal end of the bougie may be decoupled from the proximal end.

12. Mechanically-guided transoral bougie, in accordance to claim 11, wherein such decoupling is performed by increasing the diameter of the distal end of the bougie by insufflating low pressure balloons located either on the side of the bougie or these balloons may also be placed circumferentially.

13. Mechanically-guided transoral bougie, in accordance to claim 11, wherein the bougie itself acts as a stent.

14. Mechanically-guided transoral bougie, in accordance to claim 13, wherein to extract the bougie said balloons could be burst endoscopically.

15. Mechanically-guided transoral bougie, in accordance to claim 11, wherein said distal end could be made of a bioabsorbable material.

16. Mechanically-guided transoral bougie, in accordance to claim 11, wherein the bougie acts as a delivery mechanism for a stent.

17. Mechanically-guided transoral bougie, in accordance to claim 11, wherein an anastomotic device that can be draped around the outside of the bougie, or passed through the inner opening for deployment.
